(19) United States
(12) Patent Application Publication
(10) Pub. No.: US 2019/0290175 A1
(43) Pub. Date: Sep. 26, 2019

(54) BIOLOGICAL INFORMATION MEASUREMENT APPARATUS AND NON-TRANSITORY COMPUTER READABLE MEDIUM

(71) Applicant: FUJI XEROX CO., LTD., Tokyo (JP)

(72) Inventors: Hideyuki UMEKAWA, Kanagawa (JP); Manabu AKAMATSU, Kanagawa (JP); Kazuhiro SAKAI, Kanagawa (JP); Tomoaki KOJIMA, Kanagawa (JP); Hideaki OZAWA, Kanagawa (JP)

(73) Assignee: FUJI XEROX CO., LTD., Tokyo (JP)

(21) Appl. No.: 16/255,845

(22) Filed: Jan. 24, 2019

(30) Foreign Application Priority Data
Mar. 23, 2018 (JP) 2018-057120

(51) Int. Cl. A61B 5/1455; A61B 5/00; A61B 5/08

(52) U.S. Cl. CPC A61B 5/14552; A61B 5/6853; A61B 2503/40; A61B 5/746; A61B 5/0816

(57) ABSTRACT

A biological information measurement apparatus includes a first measurement unit that measures a value representing oxygen concentration in blood of a subject, and a second measurement unit that measures, by referring to a change in the value measured by the first measurement unit, as an oxygen circulation time within a predetermined time period with an end time thereof set to be later than a restart time of breathing of a subject from holding of breathing, a time duration from the restart time to a detection time of an inflection point of the value detected after the restart time of breathing.

### CROSS-REFERENCE TO RELATED APPLICATIONS

This application is based on and claims priority under 35 USC 119 from Japanese Patent Application No. 2018-057120 filed Mar. 23, 2018.

### BACKGROUND

### (i) Technical Field

The present disclosure relates to a biological information measurement apparatus and a non-transitory computer readable medium.

### (ii) Related Art

Japanese Unexamined Patent Application Publication No. 2015-190413 (the Re-publication (JP) of PCT International Publication) discloses a circulation time measurement apparatus. The circulation time measurement apparatus includes a signal acquisition unit that acquires an expiratory flow signal indicating a change in an expiratory flow with time, and an oxygen saturation signal indicating oxygen saturation changing with time, and a circulation time calculation unit that measures an oxygen circulation time of blood, based on a time difference between a first time in the expiratory flow signal and a second time in the oxygen saturation signal indicating a rise in the oxygen saturation corresponding to restart of breathing at the first time.

Measurement methods of measuring biological information using an oxygen circulation time indicating time to carry oxygen taken into a body to a predetermined location have been developed.

The oxygen circulation time is represented by a time duration from the restart of breathing of a subject from a breath hold state to an inflection point where the oxygen saturation measured at a predetermined location of the subject changes from decreasing to increasing in response to the restart of breathing.

If the measured oxygen saturation indicates an ideal change, only a single inflection point appears after the restart of breathing of the subject. The oxygen circulation time may be obtained using the appearing inflection point.

However, depending on measurement conditions of the oxygen saturation, multiple inflection points may appear after the restart of breathing. In such a case, the oxygen circulation time may be a predetermined time duration until an inflection point that appears in a predetermined order of appearance, such as the inflection point appearing first after the restart of breathing of the subject. But the inflection point appearing first may not necessarily be an inflection point caused by the restart of breathing.

### SUMMARY

Aspects of non-limiting embodiments of the present disclosure relate to a biological information measurement apparatus that increases the measurement accuracy of the oxygen circulation more than when the oxygen circulation time is measured using the inflection point in the predetermined order of appearance obtained from a value indicating the oxygen concentration in blood. Also, the aspects of non-limiting embodiments of the present disclosure relate to a non-transitory computer readable medium.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According town aspect of the present disclosure, there is provided a biological information measurement apparatus. The biological information measurement apparatus includes a first measurement unit that measures a value representing oxygen concentration in blood of a subject, and a second measurement unit that measures, by referring to a change in the value measured by the first measurement unit, as an oxygen circulation time within a predetermined time period with an end time thereof set to be later than a restart time of breathing of the subject from holding of breathing, a time duration from the restart time to a detection time of an inflection point of the value detected after the restart time of breathing.

### BRIEF DESCRIPTION OF THE DRAWINGS

Exemplary embodiments of the present disclosure will be described in detail based on the following figures, wherein:

FIG. 1 is a schematic view illustrating how oxygen saturation in blood is measured;

FIG. 2 is a graph illustrating a change in an amount of light adsorbed into a living body;

FIG. 3 illustrates an example of the adsorption amount of light on wavelengths of oxygenated hemoglobin and reduced hemoglobin;

FIG. 4 is a block diagram illustrating a biological information measurement apparatus of a first exemplary embodiment;

FIG. 5 illustrates a layout example of a light emitting element and a light receiving element;

FIG. 6 illustrates another layout example of the light emitting element and the light receiving element;

FIG. 7 illustrates an example of a respiratory waveform;

FIG. 8 illustrates an example of a change in the oxygen saturation in blood caused by the holding of breathing and the restart of breathing;

FIG. 9 illustrates an example of a change in the reciprocal of the oxygen saturation in the blood caused by the holding of breathing and the restart of breathing;

FIG. 10 is a block diagram of an electrical system of the biological information measurement apparatus;

FIG. 11 is a flowchart illustrating an example of a biological information measurement process of the first exemplary embodiment;

FIG. 12 is a flowchart illustrating the biological information measurement process performed when the biological information measurement apparatus of the first exemplary embodiment has received a modification instruction to modify an appropriate time period;

FIG. 13 is a block diagram illustrating of a biological information measurement apparatus of a second exemplary embodiment;

FIG. 14 is a flowchart illustrating a biological information measurement process of the second exemplary embodiment;

FIG. 15 is a flowchart illustrating an example of an appropriate time period updating process; and

FIG. 16 is a flowchart illustrating another example of the appropriate time period updating process.

### DETAILED DESCRIPTION

Exemplary embodiments are described below with reference to the drawings. Identical elements and operations are designated with the same reference symbols throughout the drawings, and the discussion thereof is not duplicated.

### First Exemplary Embodiment

A biological information measurement apparatus 10 measures information (biological information) related to a living body 8, in particular, biological information related to the circulatory system of the living body 8. The circulatory system generically refers to a group of organs to circulate and transport a body fluid, such as the blood in the living body 8.

There are multiple indexes in the biological information related to the circulatory system. One of the indexes indicating the state of the heart sending the blood out into a blood vessel is a cardiac output (CO) that indicates an amount of blood output from the heart.

If the cardiac output falls below a standard value, there is a possibility of a left heart failure, and if the cardiac output rises above the standard value, there is a possibility of a right heart failure. The cardiac output is used for the examination of a variety of cardiopathy patients and for the assessment of reactions of medication.

In a measurement method of the cardiac output, a catheter with a balloon is inserted into the pulmonary artery of a subject whose cardiac output is to be measured, the oxygen saturation of the blood is measured while the balloon is expanded or contracted, and then the cardiac output is calculated from the measured oxygen saturation. The oxygen saturation of the blood is one of the indexes indicating the oxygen concentration of the blood, and indicates how much of hemoglobin of the blood is linked with oxygen. As the oxygen saturation of the blood falls, the subject is likely to suffer from a symptom, such as anemia.

However in the measurement of the cardiac output using the catheter, the catheter with the balloon is to be inserted into the blood vessel of the subject, and a surgical operation is to be performed. The degree of invasiveness is thus higher than in other measurement methods.

Studies have been made to measure the cardiac output using the oxygen saturation from the pulse wave of the subject in a manner such that the burden on the subject is less than in the measurement method of the cardiac output using the catheter. The pulse wave is an index indicating a change of the blood vessel responsive to the beat when the heart outputs the blood.

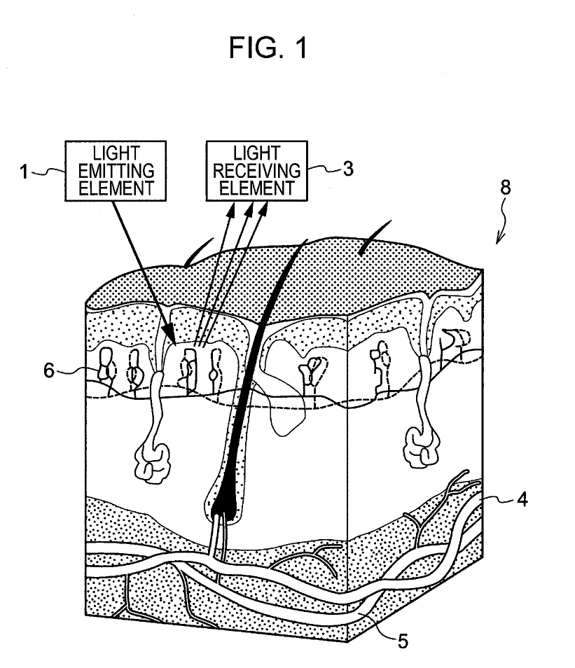

Referring to FIG. 1, the measurement method of the oxygen saturation in the blood serving as the biological information is described below with reference to FIG. 1.

Referring to FIG. 1, the oxygen saturation of the blood is measured as described below. A light emitting element 1 radiates light on the body (the living body 8) of the subject. The oxygen saturation is measured using the intensity of light reflected from or transmitted through the artery 4, the vein 5, the capillary 6, and the like running throughout the body of the subject, and then received by a light receiving element 3. More specifically, the oxygen saturation is measured using the reflected light or transmitted light.

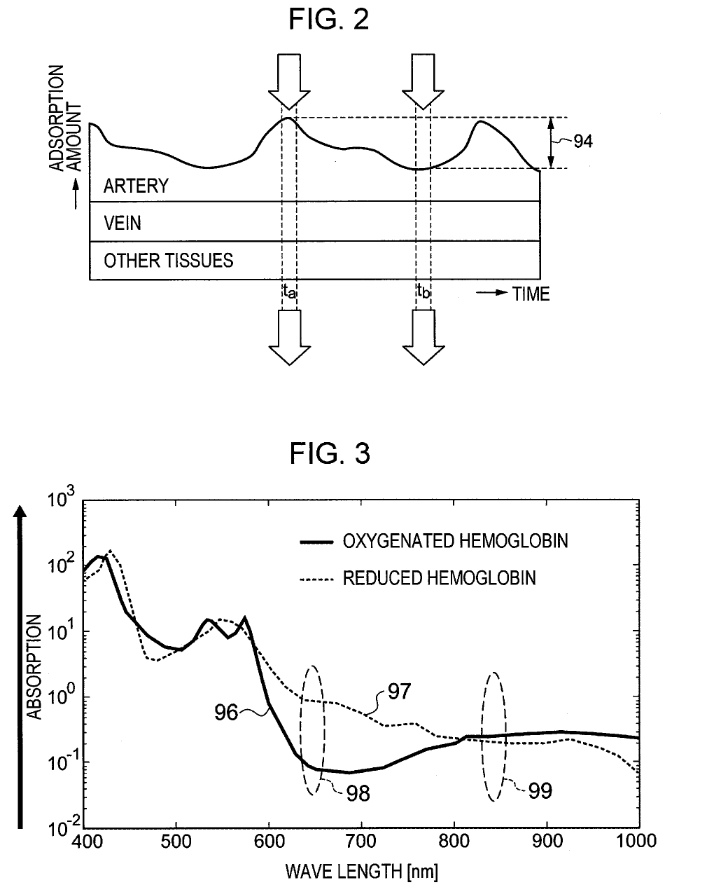

FIG. 2 illustrates a concept of an amount of light adsorbed by the living body 8. Referring to FIG. 2, the adsorption amount of the living body 8 tends to vary with time.

Concerning the detail of the change in the adsorption amount in the living body 8, it is understood that the adsorption amount varies largely depending on the artery 4 and the change in the adsorption amount on the other tissues including the vein 5 and still tissues is small enough and is considered almost unvaried in comparison with the artery 4. Since the artery blood output from the heart moves in a pulse wave through the blood vessel, the artery 4 expands or shrinks with time in the direction of cross-section of the artery 4, and varies in the thickness thereof. Referring to FIG. 2, a range labeled with a dual-headed line 94 indicates an amount of change in the adsorption amount responsive to the change in the thickness of the artery 4.

Let Iₐ represent an amount of received light at time tₐ, and let I_{b} represent an amount of received light at time t_{b}, and an amount of change ΔA in the adsorption amount of light caused in response to a change in the thickness of the artery 4 is determined in accordance with formula (1).

ΔA=In(I_{b}/Iₐ) (1)

FIG. 3 illustrates an example of the adsorption amount of light of each wave length of hemoglobin linked with oxygen flowing through the artery 4 (oxygenated hemoglobin) and hemoglobin not linked with oxygen flowing through the artery 4 (reduced hemoglobin). Referring to FIG. 3, a graph 96 represents the adsorption amount of light of the oxygenated hemoglobin and a graph 97 represents the adsorption amount of light of the reduced hemoglobin.

It is understood as illustrated in FIG. 3 that the oxygenated hemoglobin is likely to adsorb more light in an infrared (IR) region 99 having a wavelength at or close to about 850 nm than the reduced hemoglobin and that the reduced hemoglobin is likely to adsorb more light in an red region 98 having a wavelength at or close to about 660 nm in particular than the oxygenated hemoglobin.

It is also understood that the oxygen saturation is proportional to a ratio of amounts of change ΔA in the adsorption amounts of different waveforms.

A difference between the light adsorption amount of the oxygenated hemoglobin and the light adsorption amount of the reduced hemoglobin is more likely to appear in a combination of infrared (IR) light and red light than in other combinations of waveforms. Oxygen saturation S is calculated in accordance with formula (2) by calculating a ratio of an amount of change ΔA_{Red} of the adsorption amount with the living body 8 irradiated with the red light to an amount of change ΔA_{IR} of the adsorption amount with the living body 8 irradiated with the IR light. In formula (2), k represents a proportional constant.

S=k(ΔA_{Red}/ΔA_{IR}) (2)

To calculate the oxygen saturation of the blood, the living body 8 is irradiated with light rays of difference waveforms emitted from multiple light emitting elements 1. More specifically, the light emitting element 1 emitting the IR light and the light emitting element 1 emitting the red light are used for the living body 8. The time periods of light emissions of the light emitting element 1 emitting the IR light and the light emitting element 1 emitting the red light may overlap each other. But desirably, the light emitting element 1 emitting the IR light and the light emitting element 1 emitting the red light emit in non-overlapping emission time periods. The light receiving element 3 receives the reflected light or transmitted light in response to each light emitting element 1. Based on the received amount of light at light reception time points, calculation is made in accordance with formula (1), formula (2), or any formula of related art obtained by rewriting formula (1) or formula (2) to calculate the oxygen saturation.

The formula obtained by rewriting formula (1) may be formula (3) that is obtained by expanding formula (1) to determine an amount of light AA in the adsorption amount of light.

ΔA=In I_{b}−In Iₐ (3)

Formula (1) may be rewritten as formula (4) as follows:

ΔA=In(I_{b}/Iₐ)=In(1+(I_{b}−Iₐ)/Iₐ) (4)

Generally, since (I_{b}−Iₐ)<<Iₐ, In (I_{b}/Iₐ)≈(I_{b}−Iₐ)/Iₐ holds. The amount of change ΔA in the light adsorption amount may be determined using formula (5) instead of formula (1).

ΔA≈(I_{b}−Iₐ)/Iₐ (5)

If the light emitting element 1 emitting the IR light is differentiated from the light emitting element 1 emitting the red light in the following discussion, the light emitting element 1 emitting the IR light is referred to as a “light emitting element 1A”, and the light emitting element 1 emitting the red light is referred to as a “light emitting element 1B”.

Since the oxygen saturation of the blood is measured in the method described above by placing the light emitting element 1 and the light receiving element 3 closer to the body surface of the subject, the burden on the subject is lightened more than when the oxygen saturation of the blood is measured by inserting the catheter into the blood vessel.

The biological information measurement apparatus 10 calculates the cardiac output in the method described below using the measured oxygen saturation of the subject.

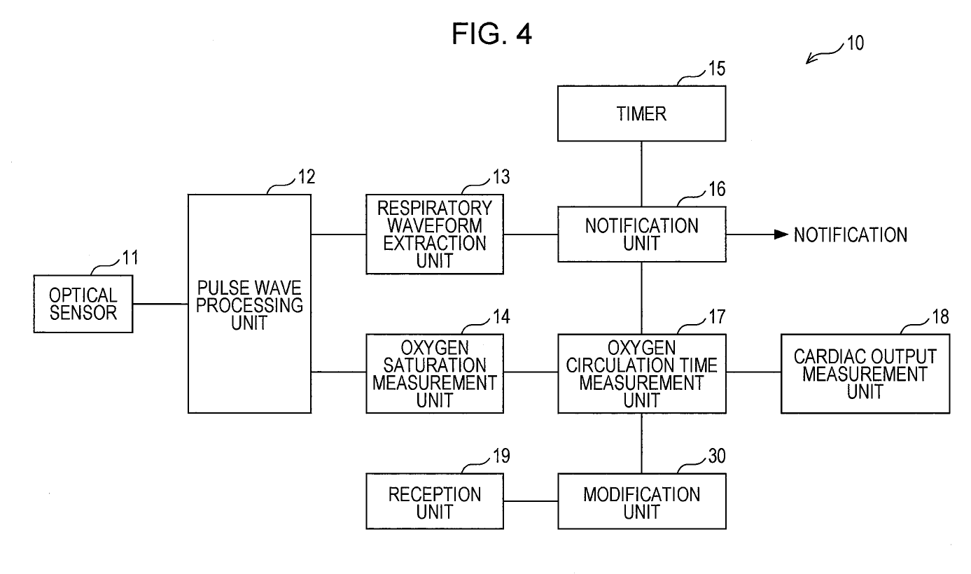

FIG. 4 is a block diagram illustrating the biological information measurement apparatus 10. Referring to FIG. 4, the biological information measurement apparatus 10 includes an optical sensor 11, a pulse wave processing unit 12, a respiratory waveform extraction unit 13, an oxygen saturation measurement unit 14, a timer 15, a notification unit 16, an oxygen circulation time measurement unit 17, a cardiac output measurement unit 18, a reception unit 19, and a modification unit 30.

The optical sensor 11 includes the light emitting element 1A emitting the IR light having a wavelength of about 850 nm as the center wavelength, the light emitting element 1B emitting the red light having a wavelength of about 660 nm as the center wavelength, and the light receiving element 3 that receives the IR light and the red light.

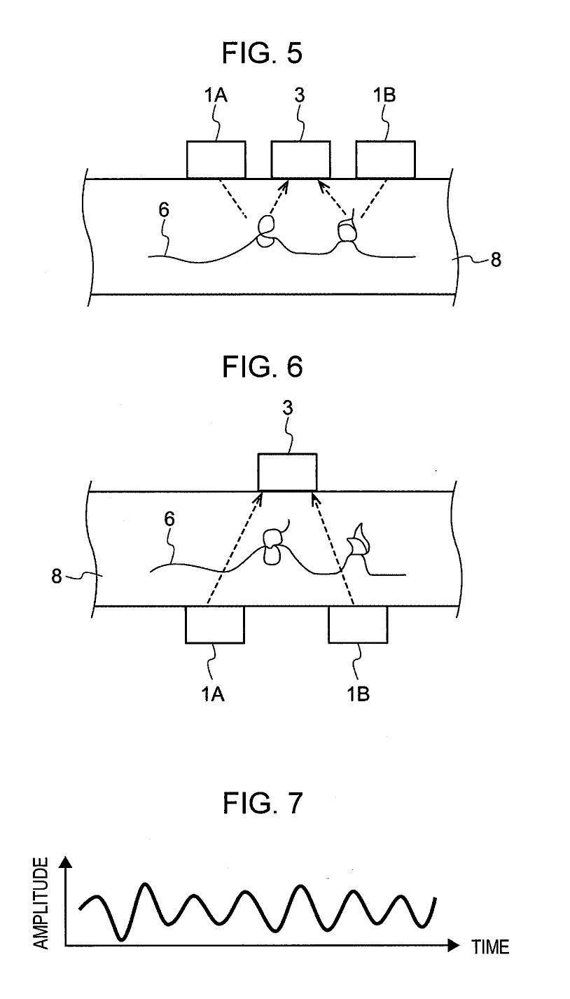

FIG. 5 illustrates the layout example of the light emitting element 1A, the light emitting element 1B, and the light receiving element 3 in the optical sensor 11. The light emitting element 1A, the light emitting element 1B, and the light receiving element 3 are laid out on one surface of the living body 8 as illustrated in FIG. 5. In such a case, the light receiving element 3 receives the IR light and the red light reflected from the capillary 6 and the like.

However, the layout of the light emitting element 1A, the light emitting element 1B, and the light receiving element 3 is not limited to the layout example of FIG. 5. For example, as illustrated in FIG. 6, the light emitting elements 1A and 1B and the light receiving element 3 may be placed in opposed positions with the living body 8 located therebetween. In such a case, the light receiving element 3 receives the IR light and the red light transmitted through the living body 8.

The light emitting element 1A and the light emitting element 1B may be a surface emitting laser, such as a vertical cavity surface emitting laser (VCSEL). The light emitting element 1A and the light emitting element 1B are not limited to a surface emitting laser, and may be an edge emitting laser. Alternatively, the light emitting element 1A and the light emitting element 1B may be light emitting diodes (LEDs).

The optical sensor 11 includes a clip (not illustrated) to mount the optical sensor 11 onto a location of the body of the subject. The optical sensor 11 is mounted on and in contact with the surface of the body of the subject with the clip (not illustrated) in a manner such that the IR light and the red light are not leaked out of the optical sensor 11. The optical sensor 11 is desirably mounted on the body surface of the subject such that the light receiving element 3 accurately receives the IR light and the red light reflected from or transmitted through the living body 8 of the subject. However, the optical sensor 11 may be spaced apart from the body surface within a range where the light receiving element 3 is still able to receive the IR light and the red light reflected from the living body 8 of the subject or the IR light and the red light transmitted through the living body 8 of the subject.

The optical sensor 11 converts the amount of light of the IR light ray and the red light ray received by the light receiving element 3 into a voltage value, and notifies the pulse wave processing unit 12 of the voltage value.

Since each of the light emitting element 1A and the light emitting element 1B emits a predetermined amount of light, the adsorption amounts of the IR light ray and the red light ray in the living body 8 are obtained from the amounts of light of the IR light ray and the red light ray received by the optical sensor 11.

Using the amounts of light of the IR light ray and the red light ray received from the optical sensor 11, the pulse wave processing unit 12 generates a pulse wave signal representing the pulse wave of the subject obtained from the IR light ray, and a pulse wave signal representing the pulse wave of the subject obtained from the red light ray. The pulse wave processing unit 12 amplifies voltage values corresponding to the received amounts of the IR light ray and the red light ray such that the amplified voltage values fall within a predetermined range appropriate for the generation of the pulse wave signal. The pulse wave processing unit 12 generates noise-free pulse wave signals using a filter of the related art.

The pulse wave processing unit 12 notifies the respiratory waveform extraction unit 13 and the oxygen saturation measurement unit 14 of the generated pulse wave signals.

Upon receiving the pulse wave signal from the pulse wave processing unit 12, the respiratory waveform extraction unit 13 extracts from the pulse wave signal a respiratory wave representing the respiratory state of the subject.

More specifically, the respiratory waveform extraction unit 13 detects a maximum value and a minimum value of one of the pulse wave signals within a predetermined time duration (1 minute, for example), obtained from the IR light ray and the pulse wave signal obtained from the red light ray. The respiratory waveform extraction unit 13 extracts the respiratory waveform of the subject from a line (peak line) that connects the detected maximum values or a line (bottom line) that connects the detected minimum values.

FIG. 7 illustrates an example of a respiratory waveform extracted from the pulse wave signal by the respiratory waveform extraction unit 13.

The respiratory waveform extraction unit 13 extracts the respiratory waveform using the pulse wave signal obtained from the IR light ray. Referring to FIG. 3, the oxygenated hemoglobin is likely to adsorb the IR light ray than the red light ray, and the amplitude of the pulse wave signal obtained from the IR light ray tends to be larger in response to a change in the width of the artery 4 than the amplitude of the pulse wave signal obtained from the red light ray. The respiratory waveform extracted from the pulse wave signal obtained from the IR light ray is more distinct in change in waveform than the respiratory waveform extracted from the pulse wave signal obtained from the red light ray. A higher accuracy respiratory waveform thus results.

The respiratory waveform extraction unit 13 refers to the respiratory waveform extracted from the pulse wave signal and then notifies the notification unit 16 of the respiratory state of the subject, such as the holding of breathing, or the restart of breathing.

The oxygen saturation measurement unit 14 is an example of a first measurement unit that measures the oxygen saturation of the subject from the pulse wave signal upon receiving the pulse wave signal from the pulse wave processing unit 12. More specifically, using the pulse wave signal, the oxygen saturation measurement unit 14 calculates, in accordance with formula (1), the amount of change ΔA_{IR} of the adsorption amount of the IR light ray and the amount of change ΔA_{Red} of the adsorption amount of the red light ray in response to the change in the thickness of the artery 4. Using the calculated amounts of change ΔA_{IR} and ΔA_{Red}, the oxygen saturation measurement unit 14 calculates the oxygen saturation of the subject in accordance with formula (2), and notifies the oxygen circulation time measurement unit 17 of the calculated oxygen saturation.

In an example described below, the oxygen saturation measurement unit 14 measures the oxygen saturation of the subject. Alternatively, the oxygen saturation measurement unit 14 may measure any value that indicates a time-series change of the oxygen saturation of the subject. For example, the oxygen saturation measurement unit 14 may measure a value correlated with the time-series change of the oxygen saturation, such as the reciprocal of the oxygen saturation, or a ratio of the amount of change ΔA_{Red} to the amount of change ΔA_{IR}.

The notification unit 16 starts up the timer 15 in response to the notification of the holding of breathing of the subject from the respiratory waveform extraction unit 13. If a breath holding period reaches a specific time duration, the notification unit 16 notifies the subject holding breath of a restart notification to restart breathing. If information that merits attention is found in the measurement of the cardiac output, the notification unit 16 issues a warning.

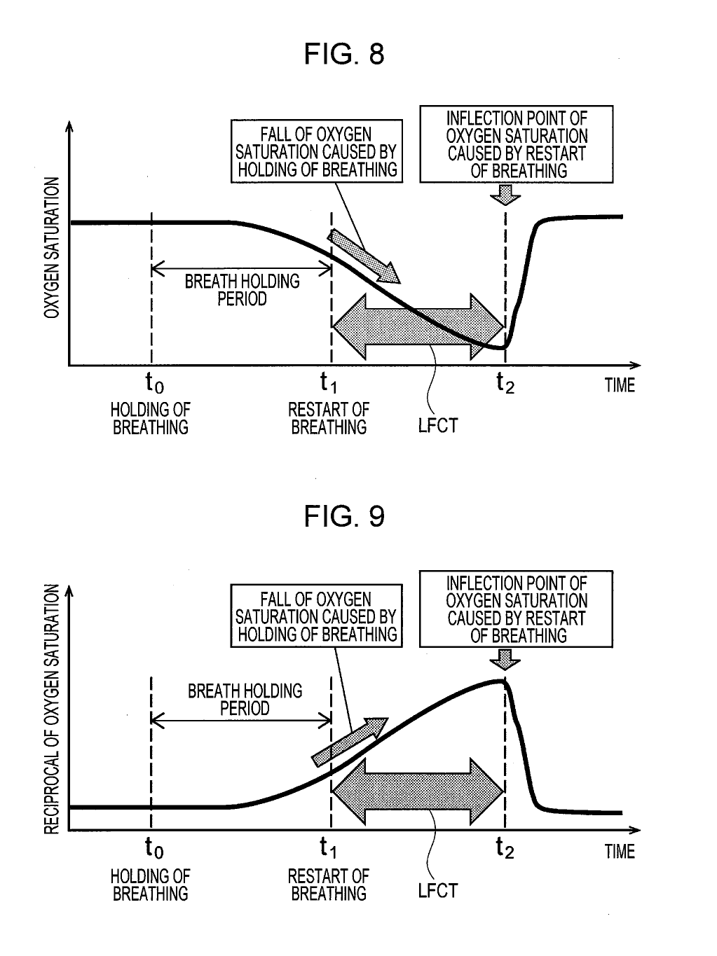

Graphs in FIG. 8 illustrate an example of changes in the oxygen saturation of the blood at a specific location of the subject. In FIG. 8, the abscissa represents time, and the ordinate represents oxygen saturation.

When the subject holds breath at time t₀, the oxygen saturation of the blood of the subject starts decreasing. When the predetermined time period determined to be a time duration throughout which the subject holds breath has elapsed (restart time t₁), the subject restarts breathing. But it takes time for oxygen taken from the lungs into the blood subsequent to the restart of breathing to travel to a predetermined location of the subject. Even subsequent to the restart time t₁, the oxygen saturation of the blood of the subject still continues to decrease. Oxygen taken into the blood from the lungs subsequent to the restart of breathing then reaches the specific location, and the oxygen saturation of the blood of the subject turns to increasing.

A point where the oxygen saturation of the blood turns from decreasing to increasing is hereinafter referred to as an “inflection point”. The point where the oxygen saturation of the blood turns from decreasing to increasing may not be represented by a single point along the waveform of the oxygen saturation, but may be expanded into a range of the waveform.

There may be multiple inflection points along the waveform representing the change in the oxygen saturation, but the number of inflection points appearing in response to the restart of breathing of the subject is one. This inflection point may be referred to as a “standard inflection point”. The inflection points other than the standard inflection point are considered to be caused when measurements different from the predetermined, ideal measurement of the oxygen saturation are performed.

More specifically, the causes for the changes in the oxygen saturation include a change in the measurement environment, such as a change in a contact state of the optical sensor 11, and a psychological or physical change of the subject, such nervousness of the subject. Furthermore, the causes for the changes in the oxygen saturation may include an external disturbance, such as a measurement error of the optical sensor 11 caused by the effect of the sun light, and a change in the stability of the respiratory state, such as re-holding breath subsequent to the restart of breathing performed before the elapse of a specified time duration.

If the standard inflection point where the oxygen saturation turns from decreasing to increasing after the subject restarts breathing is detected at detection time t₂, an oxygen circulation time is represented by a difference between restart time t₁ and detection time t₂.

The oxygen circulation time represents time used to transport oxygen from the lungs to a specific location, and is also referred to as an “oxygen transport time”.

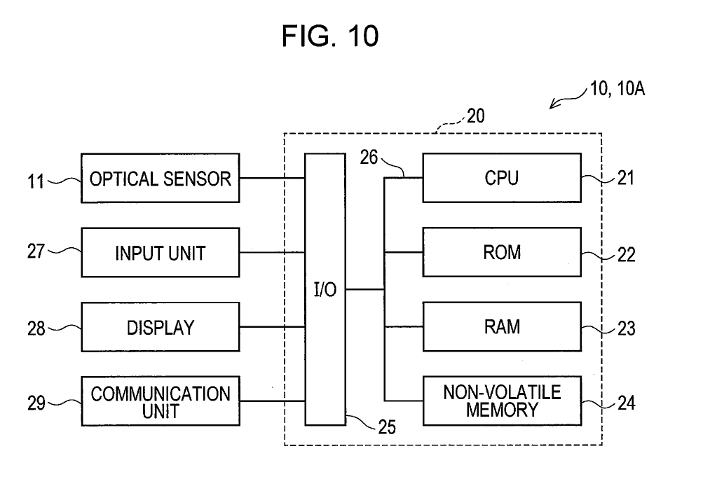

If the reciprocal of the oxygen saturation is measured by the oxygen saturation measurement unit 14, the waveform of the oxygen saturation of the blood at the specific location of the subject is obtained as illustrated in FIG. 9 by tracing the waveform of FIG. 8 upside down. In this case, as well, the oxygen circulation time is the difference between restart time t₁ and detection time t₂.

As long as the inflection point where the oxygen saturation turns from decreasing to increasing is traced in time sequence, the oxygen saturation measurement unit 14 may express the oxygen saturation in any form.

Since the oxygen circulation time determined from the oxygen saturation tends to vary in accuracy depending the variation in the length of the breath holding period, a specific time that determines the breath holding period is set up. The specific time has a value that is determined in advance through computer simulation, based on experiments of the real machine of the biological information measurement apparatus 10 or design specifications of the biological information measurement apparatus 10, such that the measurement accuracy of the oxygen circulation time provided by the biological information measurement apparatus 10 increases.

The oxygen circulation time measurement unit 17 receives from the notification unit 16 information that the subject restarts breathing, and then stores as the restart time t₁ the time when the information on the restart of breathing is received. The oxygen circulation time measurement unit 17 monitors the oxygen saturation measured by the oxygen saturation measurement unit 14 to detect the inflection point of the oxygen saturation. The oxygen circulation time measurement unit 17 stores as the detection time t₂ the time when the inflection point of the oxygen saturation is detected. The oxygen circulation time measurement unit 17 thus measures as the oxygen circulation time the difference between the restart time t₁ and the detection time t₂.

Note that the oxygen circulation time measurement unit 17 determines as the detection time t₂ of the standard inflection point, the detection time of the inflection point detected later than the restart time t₁ and falling within the predetermined time period subsequent to the restart time t₁ when the subject restarts breathing. More specifically, if the inflection point is detected outside the predetermined time period that is set up to include a time duration subsequent to the restart time t₁ or if the inflection point is detected prior to the restart time t₁ during the predetermined time period, the oxygen circulation time measurement unit 17 determines that such an inflection point is not the standard inflection point.

Since it takes time for oxygen taken into the blood from the lungs to reach a specific location in response to the restart of breathing, the standard inflection point appears later than the restart time t₁ when the subject restarts breathing. The time duration that oxygen takes to travel from the lungs to the specific location is subject to an upper limit value that is determined from a medical point of view. The predetermined time period thus includes a time duration within which the standard inflection point falls. The end time of the predetermined time period is naturally set to be later than the restart time t₁ when the subject restarts breathing.

The condition that the inflection point falls within the predetermined time period is to be satisfied to determine that the detected inflection point is the standard inflection point. In the following discussion, the predetermined time period is referred to as an “appropriate time period”.

The start time of the appropriate time period is not limited to any time. Since the standard inflection point appears later than the restart time t₁ when the subject restarts breathing, the start time of the appropriate time period is desirably set to be later than the restart time t₁. The start time of the appropriate time period comes naturally prior to the end time of the appropriate time period. The inflection point falling within the appropriate time period is determined to be the standard inflection point without comparing the detection time of the inflection point with the restart time t₁. In an example described below, the start time of the appropriate time period is set to be later than the restart time t₁.

Since an inflection point that appears during a time duration between the restart of breathing of the subject and the start time of the appropriate time period is not regarded as the standard inflection point, the time duration is thus referred to as a “waiting time”.

The oxygen circulation time measurement unit 17 notifies the cardiac output measurement unit 18 of the measured oxygen circulation time. The oxygen circulation time measurement unit 17 is an example of a second measurement unit that measures the oxygen circulation time.

The measurement location of the oxygen circulation time is determined, based on the mounting location of the optical sensor 11 on the subject. In accordance with the first exemplary embodiment, the oxygen circulation time is measured with oxygen traveling from the lungs to the tip of a finger when the optical sensor 11 is mounted on the tip of the finger. In this setting, the distance from the lungs to the tip of the finger is longer than the distance from the lungs to other locations of the subject. A longer oxygen circulation time thus results. A more accurate oxygen circulation time results than when the optical sensor 11 is mounted on the other locations of the body of the subject.

The oxygen circulation time from the lungs to the tip of the finger is occasionally referred to as a lung to finger circulation time (LFCT). In accordance with the first exemplary embodiment, the optical sensor 11 is mounted on the tip of the finger, and the oxygen circulation time measurement unit 17 measures LFCT. The mounting location of the optical sensor 11 is not limited to the tip of the finger. The optical sensor 11 may be mounted on any location of the body of the subject as long as the measurement error in the obtained oxygen circulation time falls within a predetermined range. For example, an example of the mounting location may be the neck, the shoulder, a peripheral portion of the subject. The tip of the finger refers to the tip of the finger of the subject's hand. Alternatively, the optical sensor 11 may be mounted on the tip of a toe.

The cardiac output measurement unit 18 measures the cardiac output of the subject using LFCT received from the oxygen circulation time measurement unit 17.

The cardiac output CO is determined from LFCT in accordance with formula (6) of the related art.

CO=(a0×S)/LFCT (6)

where a0 is a constant. For example, a0=50. S represents the body surface area of the subject (with unit being m²), and LFCT is with unit being second.

The cardiac output measurement unit 18 may measure information related to the cardiac output in addition to the cardiac output. The “information related to the cardiac output” refers to information that is correlated with the cardiac output. For example, the information related to the cardiac output may be a cardiac index or a single cardiac output.

The “cardiac index” corrects a difference between the cardiac outputs caused by a difference in physique, and is a value that is obtained by dividing the cardiac output of the subject by the body surface area of the subject. The “single cardiac output” is a value indicating an amount of blood output to the artery 4 when the heart shrinks once, and is determined by dividing the cardiac output by the heart rate per minute.

The reception unit 19 is an example of a reception unit that receives an instruction from users of the biological information measurement apparatus 10 via an input unit 27 to be discussed below. The “users of the biological information measurement apparatus 10” include at least one person concerned, including the subject, a user, such as a medical practitioner who measures biological information of the subject, and an administrator who maintains the biological information measurement apparatus 10.

Instructions received by the reception unit 19 include a modification instruction to modify the length of the appropriate time period, namely, at least one of the start time and the end time of the appropriate time period, and a measurement instruction to measure the cardiac output by starting measuring the oxygen saturation of the subject.

If the instruction received by the reception unit 19 is the modification instruction to modify the appropriate time period, the modification unit 30 modifies the appropriate time period such that at least one of the start time and the end time of the appropriate time period is the value set in response to the modification instruction.

No limit is set on the start time and the end time of the appropriate time period that is to be modified in response to the modification instruction. The reception unit 19 receives the modification instruction to modify the appropriate time period such that at least the start time of the appropriate time period is later than the restart time t₁ when the subject restarts breathing, and the modification unit 30 modifies the start time of the appropriate time period to the value described in the modification instruction.

The start time and the end time of the appropriate time period may be represented by a relative time with respect to the restart time t₁ when the subject restarts breathing. In such a case, the start time or the end time of the appropriate time period set to be earlier than the restart time t₁ is a negative value, the start time or the end time of the appropriate time period set to be equal to the restart time t₁ is “0”, and the start time or the end time of the appropriate time period set to be later than the restart time t₁ is a positive value.

The modification instruction to modify the start time and the end time of the appropriate time period is not limited to the method described above. For example, the modification instruction may instruct the length of the waiting time and the length of the appropriate time period to be appropriate values. If the restart time t₁ when the subject restarts breathing is planned to be absolute time, the start time and the end time of the appropriate time period may be also indicated as absolute time.

If the appropriate time period modified in response to the modification instruction is inappropriate for the measurement of the oxygen circulation time, for example, if the end time of the appropriate time period is prior to the restart time t₁ of the appropriate time period received by the reception unit 19, the notification unit 16 serving as an example of a notification unit issues a warning to prompt the user of the biological information measurement apparatus 10 to modify the appropriate time period.

If the modification unit 30 has modified the predetermined appropriate time period, the oxygen circulation time measurement unit 17 measures LFCT by detecting the standard inflection point using the modified appropriate time period.

The biological information measurement apparatus 10 includes a computer 20. FIG. 10 is a block diagram of the electrical system of the biological information measurement apparatus 10 including the computer 20.

The computer 20 includes a central processing unit (CPU) 21, a read-only memory (ROM) 22, a random-access memory (RAM) 23, a non-volatile memory 24, and an input and output (I/O) interface 25. The CPU 21, the ROM 22, the RAM 23, the non-volatile memory 24, and the I/O interface 25 are interconnected to each other via a bus 26. The operating system used in the computer 20 is not limited to any particular operating system.

The non-volatile memory 24 is an example of a memory, and keeps storing information even when power to the non-volatile memory 24 is interrupted. For example, the non-volatile memory 24 may be a semiconductor memory or a hard disk.

The I/O interface 25 is connected to the optical sensor 11, an input unit 27, a display 28, and a communication unit 29.

The optical sensor 11 is wiredly or wirelessly connected to the I/O interface 25. The biological information measurement apparatus 10 and the optical sensor 11 may be configured to be separate units. Alternatively, the biological information measurement apparatus 10 and the optical sensor 11 may be accommodated into a unitary housing.

The input unit 27 notifies the CPU 21 of an instruction of the user of the biological information measurement apparatus 10. The input unit 27 may include a button, a touch panel, a keyboard, a mouse, and the like.

The display 28 visually displays information processed by the CPU 21 to the user of the biological information measurement apparatus 10. The display 28 may be a liquid-crystal display, an organic electroluminescent display, a projector, or the like.

The display 28 is not necessarily included in the biological information measurement apparatus 10. Any type of the display 28 may be connected to the I/O interface 25 as long as the display 28 notifies the user of the biological information measurement apparatus 10 of information notified by the biological information measurement apparatus 10, for a notification of the restart of breathing, or a warning.

If the user of the biological information measurement apparatus 10 is audibly notified of the information from the biological information measurement apparatus 10, a speaker unit in place of the display 28 may be connected to the I/O interface 25. If the user of the biological information measurement apparatus 10 is notified of the information from the biological information measurement apparatus 10 in a tentacular way, a vibration unit in place of the display 28 may be connected to the I/O interface 25. Alternatively, using multiple units including the display 28 and the speaker unit, the user of the biological information measurement apparatus 10 is notified of the information from the biological information measurement apparatus 10.

The communication unit 29 supports a communication protocol that connects the biological information measurement apparatus 10 to a communication network, such as the Internet, and performs data communication with another external apparatus connected to the communication network. The communication unit 29 may be wiredly or wirelessly connected to the communication network. If the biological information measurement apparatus 10 is not connected to the external apparatus connected to the communication network, the communication unit 29 may not necessarily be connected to the I/O interface 25.

The units connected to the I/O interface 25 are not limited to those described above. For example, a printer may be connected to the I/O interface 25.

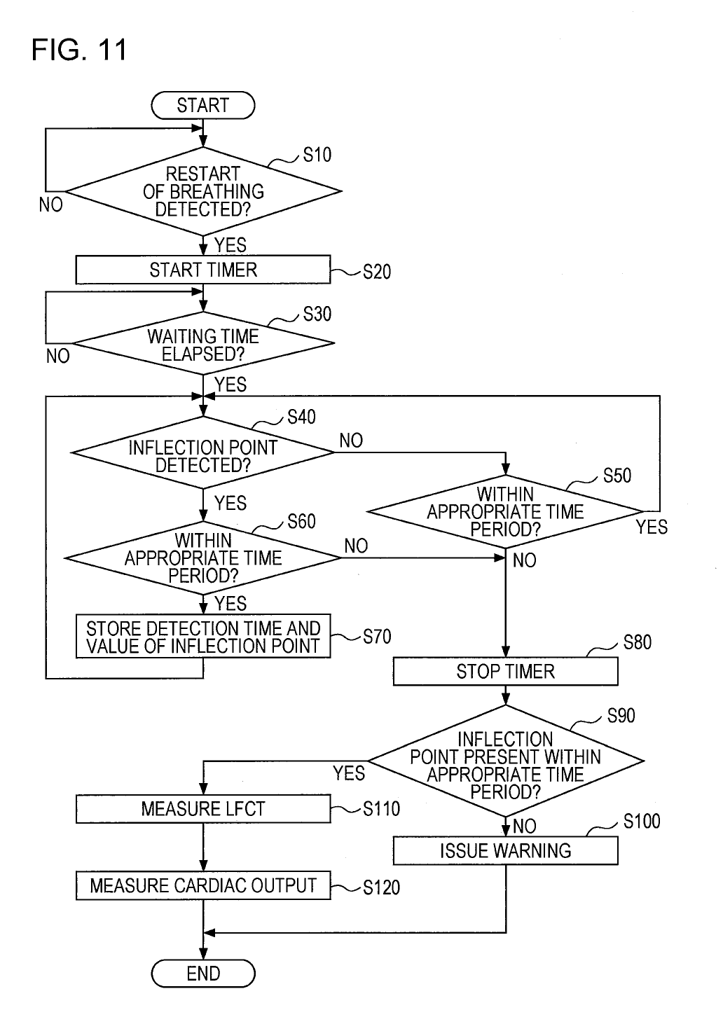

The process performed by the biological information measurement apparatus 10 is described below in connection with FIG. 11.

FIG. 11 is a flowchart illustrating a biological information measurement process performed by the CPU 21 when a measurement instruction to measure the cardiac output is received from the user of the biological information measurement apparatus 10 via the input unit 27 with the optical sensor 11 mounted on the tip of a finger of the subject. The biological information measurement apparatus 10, when receiving the measurement instruction to measure the cardiac output, measures the oxygen saturation of the subject and continues to measure until at least the end of the measurement of the cardiac output.

A biological information measurement program describing the biological information measurement process is pre-stored on the ROM 22 in the biological information measurement apparatus 10. The CPU 21 in the biological information measurement apparatus 10 reads the biological information measurement program stored on the ROM 22, and performs the biological information measurement process. The start time and the end time of the appropriate time period are represented by relative time with respect to the restart time t₁ when the subject restarts breathing, and are pre-stored on the non-volatile memory 24.

In step S10, the CPU 21 determines whether the subject having held breath for the specific time in the breath holding state restarts breathing in response to the restart notification of breathing from the biological information measurement apparatus 10.

A determination as to whether the subject has restarted breathing is made by referring to the respiratory waveform obtained from the pulse wave signal detected by the optical sensor 11. If it is determined that the subject has not restarted breathing yet, the operation in step S10 is repeatedly performed to monitor the respiratory waveform of the subject. Processing proceeds to step S20 if it is determined that the subject has restarted breathing.

The detection method of the respiratory state of the subject is not limited to referring to the respiratory waveform. For example, an air flow sensor detecting the flow of air or an air volume sensor detecting the volume of air may be mounted on the muzzle of the subject, the breathing may be directly detected. When the subject breathes, air warmed up inside the body of the subject is discharged. A temperature sensor may be mounted on the muzzle of the subject to directly detect the breathing of the subject. The chest of the subject varies in position when the subject breathes. A video of the chest of the subject captured by a camera may be analyzed, or a displacement sensor may be mounted on the chest of the subject. The breathing of the subject is thus detected.

As described above, the detection methods of detecting the respiratory state of the subject use a variety of sensors including the optical sensor 11, and the air flow sensor. Alternatively, the subject may press a button included in the input unit 27 to notify the CPU 21 of the restart of breathing. Alternatively, the timer 15 may be used to measure the specific time that describes the breath holding period. When a time-out notification given by the timer 15 to notify of the elapse of the specific time is issued, it may be determined that the subject has restarted breathing.

In step S20, the CPU 21 starts the timer 15 in synchronization with the restart of breathing of the subject. More specifically, the value on the timer 15 indicates relative time from the restart time t₁ when the subject restarts breathing.

The CPU 21 may start the timer 15 in synchronization with the holding of breathing prior to the restart of breathing of the subject. In such a case, the startup of the timer 15 in step S20 is not performed. A value that is obtained by subtracting the specific time from the value on the timer 15 represents the relative time with respect to the restart time t₁ when the subject has restarted breathing.

In step S30, the CPU 21 determines whether the value on the timer 15 has reached the start time of the appropriate time period, namely, determines whether the elapsed time from the restart of breathing of the subject has become equal to the waiting time. If the elapsed time has not become equal to the waiting time, the CPU 21 monitors the value on the timer 15 until the elapsed time becomes equal to the waiting time. On the other hand, if the elapsed time has become equal to the waiting time, processing proceeds to step S40.

In step S40, the CPU 21 detects the inflection point by referring to the oxygen saturation of the subject currently being measured. If no inflection point is detected from the oxygen saturation, processing proceeds to step S50.

In step S50, the CPU 21 determines whether the value on the timer 15 has reached the end time of the appropriate time period, namely, determines whether an inflection point has been detected within the appropriate time period. If an inflection point is detected within the appropriate time period, processing returns to step S40 to detect an inflection point of the oxygen saturation again.

If the determination operation in step S40 is affirmative, namely, if an inflection point has been detected in the oxygen saturation, processing proceeds to step S60.

In step S60, the CPU 21 refers to the timer 15 and determines again whether the inflection point detected in step S40 falls within the appropriate time period. If the inflection point is detected close to the end time of the appropriate time period, the inflection point detected in step S40 may not be the inflection point falling within the appropriate time period, depending on the detection time of the inflection point.

If the inflection point detected in step S40 is the one falling within the appropriate time period in the determination operation in step S60, processing proceeds to step S70.

In step S70, the CPU 21 stores on the RAM 23 the detection time of the inflection point detected in step S40 and the value of the oxygen saturation at the inflection point. The CPU 21 returns to step S40 and continues to detect an inflection point. In this way, the detection time and the value of the inflection point detected within the appropriate time period are stored on the RAM 23. The storage destination of the detection time and the value of the inflection point is not limited to the RAM 23. For example, the detection time and the value of the inflection point detected within the appropriate time period may be stored on a memory in an external apparatus connected to the communication network. The detection time of the inflection point is the value on the timer 15 when the inflection point is detected.

If the value on the timer 15 reaches the end time of the appropriate time period in the determination operation in step S50, or if it is determined in the determination operation in step S60 that the inflection point detected in step S40 does not fall within the appropriate time period, processing proceeds to step S80.

In step S80, the CPU 21 stops the timer 15 started up in step S20.

In step S90, the CPU 21 determines whether there is an inflection point detected within the appropriate time period and stored on the RAM 23 in step S70. If there is no inflection point detected within the appropriate time period, processing proceeds to step S100.

In such a case, LFCT of the subject is not obtained. The CPU 21 displays on the display 28 a warning message that LFCT of the subject has not been measured.

If the speaker unit is connected to the I/O interface 25, the CPU 21 may output a voice warning from the speaker unit. If the vibration unit is connected to the I/O interface 25, the CPU 21 causes the vibration unit to vibrate, and thus gives the user of the biological information measurement apparatus 10 a warning in a tentacular way. The biological information measurement process of FIG. 11 is thus complete.

If it is determined in the determination operation in step S90 that an inflection point detected within the appropriate time period is present, processing proceeds to step S110.

In step S110, the CPU 21 obtains the detection time t₂ of the inflection point stored on the RAM 23 in step S70, and causes the detection time t₂ to be re-stored on the non-volatile memory 24 such that the detection time t₂ remains stored even when power is interrupted. The detection time t₂ of the inflection point is relative time with respect to the restart time t₁ when the subject restarts breathing, and thus represents LFCT.

If multiple inflection points are detected within the appropriate time period, the detection time t₂ when the inflection point having the lowest value of the oxygen saturation, from among the inflection points, is set to be LFCT. The standard inflection point appears when the subject restarts breathing after the breath holding period. The standard inflection point thus appears with the oxygen saturation reduced most. The value of the oxygen saturation at the standard inflection point is likely to fall below the value of the oxygen saturation of another inflection point.

In step S120, the CPU 21 measures the cardiac output in accordance with formula (6) using LFCT obtained in step S110. The CPU 21 may determine information related to the cardiac output using the measured cardiac output. The biological information measurement process of FIG. 11 is thus complete.

Referring to FIG. 11, LFCT is measured from the inflection point detected within the predetermined appropriate time period. The biological information measurement apparatus 10 may be configured to modify at least one of the start time and the end time of the appropriate time period.

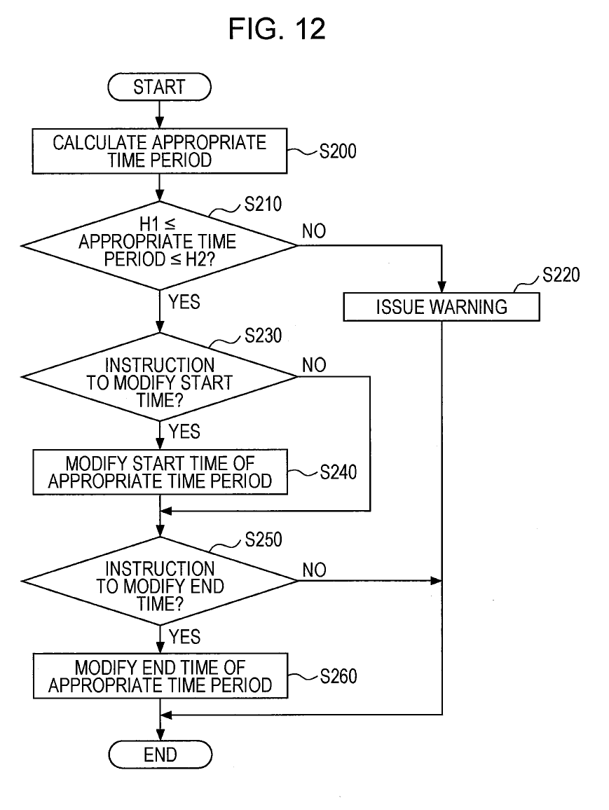

FIG. 12 is a flowchart illustrating the biological information measurement process performed by the CPU 21 when the modification instruction to modify the appropriate time period is received.

The biological information measurement program describing the biological information measurement process of FIG. 12 is pre-stored on the ROM 22 in the biological information measurement apparatus 10. The CPU 21 in the biological information measurement apparatus 10 reads the biological information measurement program from the ROM 22, and performs the biological information measurement process.

If the appropriate time period is modified in response to the modification instruction, the CPU 21 calculates the modified appropriate time period in step S200. As previously described, the modification of the appropriate time period may be performed by providing the modification instruction to modify at least one of the length of the waiting time and the length of the appropriate time period. In the operation example described here, the appropriate time period is modified by providing the modification instruction to modify at least one of the start time and the end time of the appropriate time period.

When the modification instruction to modify the start time and the end time of the appropriate time period has been provided, the CPU 21 calculates the appropriate time period from the start time and the end time described in the modification instruction. If the modification instruction to modify one of the start time and the end time of the appropriate time period is provided, the CPU 21 calculates the modified appropriate time period from one of the start time and the end time described in the modification instruction, and one of the start time and the end time not described in the present modification instruction.

If the length of the appropriate time period is much shorter than a predetermined lower limit value H₁, the standard inflection point may not be detected in the appropriate time period. If the length of the appropriate time period is much longer than a predetermined upper limit value H₂, the detection of inflection points continues even after the standard inflection point appears. A longer time may be taken before the start of the measurement of LFCT.

In step S210, the CPU 21 determines whether the length of the appropriate time period calculated in step S200 falls within a range determined by the lower limit value H₁ or above to the upper limit value H₂ or below. The lower limit value H₁ and the upper limit value H₂ are reference values defining the length of the appropriate time period suitable for detecting the standard inflection point, and are values that are determined in advance through computer simulation, based on experiments of the real machine of the biological information measurement apparatus 10 or design specifications of the biological information measurement apparatus 10. The lower limit value H₁ and the upper limit value H₂ are pre-stored on the non-volatile memory 24.

If the length of the appropriate time period is shorter than the lower limit value H₁ or longer than the upper limit value H₂, processing proceeds to step S220.

In step S220, the CPU 21 displays a warning on the display 28. The warning is displayed to notify the user of the biological information measurement apparatus 10 that if the appropriate time period is modified in accordance with the modification instruction, the length of the modified appropriate time period becomes inappropriate for the measurement of LFCT. The biological information measurement process of FIG. 12 is thus complete.

The user of the biological information measurement apparatus 10 having received the warning is thus prompted to carry out again the modification instruction modify the appropriate time period to be within the range determined by the lower limit value H₁ or above to the upper limit value H₂ or below.

If the CPU 21 determines in step S210 that the length of the appropriate time period calculated in step S200 is within the range determined by the lower limit value H₁ or above to the upper limit value H₂ or below, processing proceeds to step S230.

The length of the modified appropriate time period is thus appropriate for LFCT. Subsequent to step S230, the CPU 21 modifies the appropriate time period in response to the modification instruction.

In step S230, the CPU 21 determines whether the modification instruction to modify the start time of the appropriate time period is provided. If the modification instruction to modify the start time of the appropriate time period is provided, processing proceeds to step S240.

In step S240, the CPU 21 modifies the start time of the appropriate time period to the start time described in the modification instruction.

If it is determined in the operation in step S230 that the instruction to modify the start time of the appropriate time period is not provided, processing proceeds to step S250 with step S240 skipped.

In step S250, the CPU 21 determines whether the modification instruction to modify the end time of the appropriate time period is provided. If the modification instruction to modify the end time of the appropriate time period is provided, processing proceeds to step S260.

In step S260, the CPU 21 modifies the end time of the appropriate time period to the end time described in the modification instruction. The biological information measurement process of FIG. 12 is thus complete.

If the CPU 21 determines in the determination operation in step S250 that the instruction to modify the end time of the appropriate time period is not provided, the CPU 21 completes the biological information measurement process of FIG. 12 without performing the operation in step S260.

Verification items that determine whether the appropriate time period modified in response to the modification instruction is appropriate for measuring LFCT are not limited to the length of the appropriate time period. For example, since the standard inflection point does not appear before the subject restarts breathing, the CPU 21 may determine whether the start time of the appropriate time period is going to be modified to be prior to the restart of breathing of the subject. If the CPU 21 determines that the start time of the appropriate time period is going to be modified to be prior to the restart of breathing of the subject, the CPU 21 may issue a warning. Also, if the CPU 21 determines that the end time of the appropriate time period is going to be modified to be prior to the restart of breathing of the subject, the CPU 21 may issue a warning.

The modification instruction to modify the length of the appropriate time period may be provided instead of the modification instruction to modify at least one of the start time and the end time of the appropriate time period. In such a case, the CPU 21 may determine in step S210 that the length of the appropriate time period described in step S210 falls within the range determined by the lower limit value H₁ or above to the upper limit value H₂ or below. If the length of the appropriate time period described in step S210 falls within the range determined by the lower limit value H₁ or above to the upper limit value H₂ or below, the CPU 21 simply modifies the current length of the appropriate time period to the length of the appropriate time period described in the instruction instead of performing the operations in steps S230 through S260. Otherwise, the CPU 21 issues the warning.

If the modification instruction to modify the length of the waiting time is provided, the CPU 21 determines in step S210 whether the length of the waiting time falls within a range determined by a lower limit value J₁ or above to an upper limit value J₂ or below.

The lower limit value J₁ and the upper limit value J₂ are reference values describing the start time of the waiting time appropriate for detecting the standard inflection point, and are predetermined through computer simulation, based on experiments of the real machine of the biological information measurement apparatus 10 or design specifications of the biological information measurement apparatus 10.

It takes time for oxygen taken into the blood from the lungs to reach a specific location (a finger in this case). If the length of the waiting time becomes shorter than the lower limit value J₁, the inflection point detection is performed beyond the range where no standard inflection point appears. If the waiting time is set to be too long, the start of the inflection point detection is delayed. The length of the waiting time becomes longer than the upper limit value J₂, causing difficulty in detecting the standard inflection point.

If the CPU 21 determines that the appropriate time period falls within the range determined by the lower limit value J₁ or above to the upper limit value J₂ or below, the CPU 21 simply modifies the current length of the waiting time to the length of the waiting time described in the instruction instead of performing the operations in steps S230 through S260. Otherwise, the CPU 21 issues the warning.

The modification of the appropriate time period is described in connection with FIG. 12. The biological information measurement apparatus 10 may also receive from the user of the biological information measurement apparatus 10 the modification instruction to modify detection time of the inflection point, and may modify the detection time period of the inflection point.

In the process of the biological information measurement apparatus 10 of FIG. 11, the inflection point is detected throughout the appropriate time period from the start time to end time thereof. The detection time period of the inflection point does not necessarily have to match the appropriate time period. For example, while the oxygen saturation is measured in response to the received measurement instruction to measure the cardiac output, the inflection point of the oxygen saturation continues to be detected. From among the detected inflection points, LFCT is measured with the inflection point within the appropriate time period serving as the standard inflection point.

The appropriate time period associated with each subject may be stored on the non-volatile memory 24 in the biological information measurement apparatus 10. Using the appropriate time period associated with each subject, the CPU 21 may determine whether the inflection point of the oxygen saturation is the standard inflection point. In such a case as well, at least one of the start time and the end time of the appropriate time period associated with each subject is modified in accordance with the flowchart of FIG. 12.

Identification information identifying each subject, such as a user name and a password, is associated with the subject. The appropriate time period for each subject is stored in association with the user name and password of the subject on the non-volatile memory 24 in the biological information measurement apparatus 10. The biological information measurement apparatus 10 obtains the appropriate time period corresponding to the subject by retrieving from the non-volatile memory 24 the appropriate time period associated with the user name and password matching the user name and password received via the input unit 27.

The detection time period of the inflection point may be predetermined in the same way as the appropriate time period. At least one of the start time and the end time of the detection time period set on each subject is modified on a per subject basis in accordance with the flowchart of FIG. 12.

The biological information measurement apparatus 10 of the first exemplary embodiment measures LFCT, based on the standard inflection point. The standard inflection point is an inflection point detected with the predetermined appropriate time period, from among the inflection points representing the oxygen saturation of the blood of the subject.

### Second Exemplary Embodiment

If the biological information measurement apparatus 10 of the first exemplary embodiment is not able detect the inflection point within the set appropriate time period, the user of the biological information measurement apparatus 10 modifies the appropriate time period while predicting the timing when an inflection point appears.

A second exemplary embodiment is described below. The biological information measurement apparatus 10A of the second exemplary embodiment autonomously modifies the appropriate time period in a relatively short period of time in a manner such that an inflection point falls within the appropriate time period.

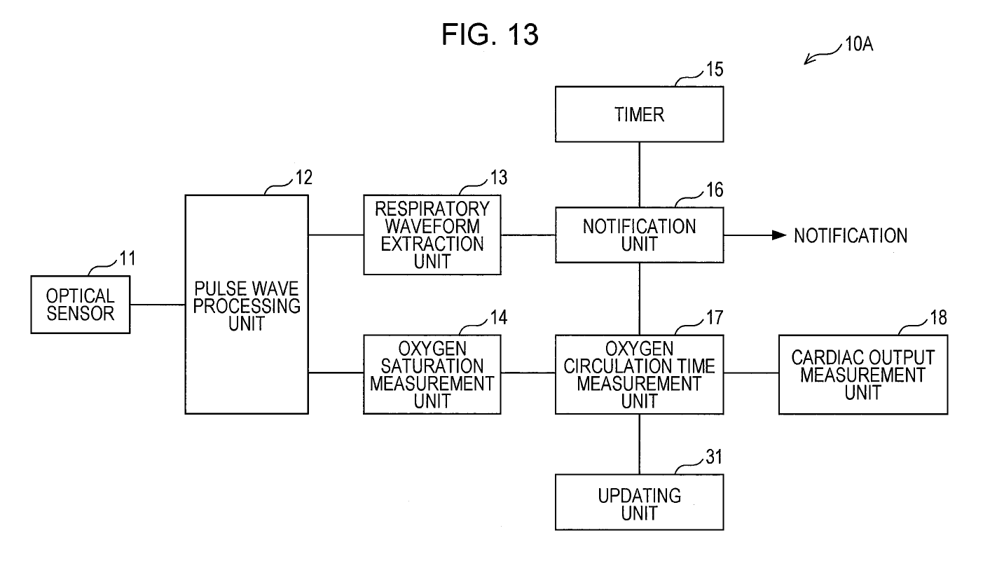

FIG. 13 illustrates the configuration of the biological information measurement apparatus 10A of the second exemplary embodiment. The biological information measurement apparatus 10A of FIG. 13 is different in configuration from the biological information measurement apparatus 10 of the first exemplary embodiment in that the biological information measurement apparatus 10A includes an updating unit 31 but is free from the reception unit 19 and the modification unit 30. The rest of the biological information measurement apparatus 10A remains unchanged from the biological information measurement apparatus 10.

The updating unit 31 refers to past LFCT measured by the biological information measurement apparatus 10A and stored on the non-volatile memory 24, namely, the detection time t₂ of the standard inflection point. As the number of measurements of LFCT increases, the detection time t₂ of the standard inflection point is more likely to fall within a particular range. If a range including all detection times t₂ is set to be the appropriate time period in a period of time as short as possible, an optimum appropriate time period estimated from the past measurements of LFCT is set.

However, LFCT varies from subject to subject, and the inflection point may not necessarily be detected within the appropriate time period set in a manner described above. The updating unit 31 updates the appropriate time period such that the appropriate time period becomes as short as possible but still includes the detection time t₂. If no inflection point is detected within the appropriate time period, the updating unit 31 updates the appropriate time period such that the appropriate time period becomes longer than the present appropriate time period. The updating unit 31 is an example of an updating unit of the second exemplary embodiment.

The electrical system of the biological information measurement apparatus 10A is identical to the electrical system of the biological information measurement apparatus 10 of the first exemplary embodiment illustrated in FIG. 10.

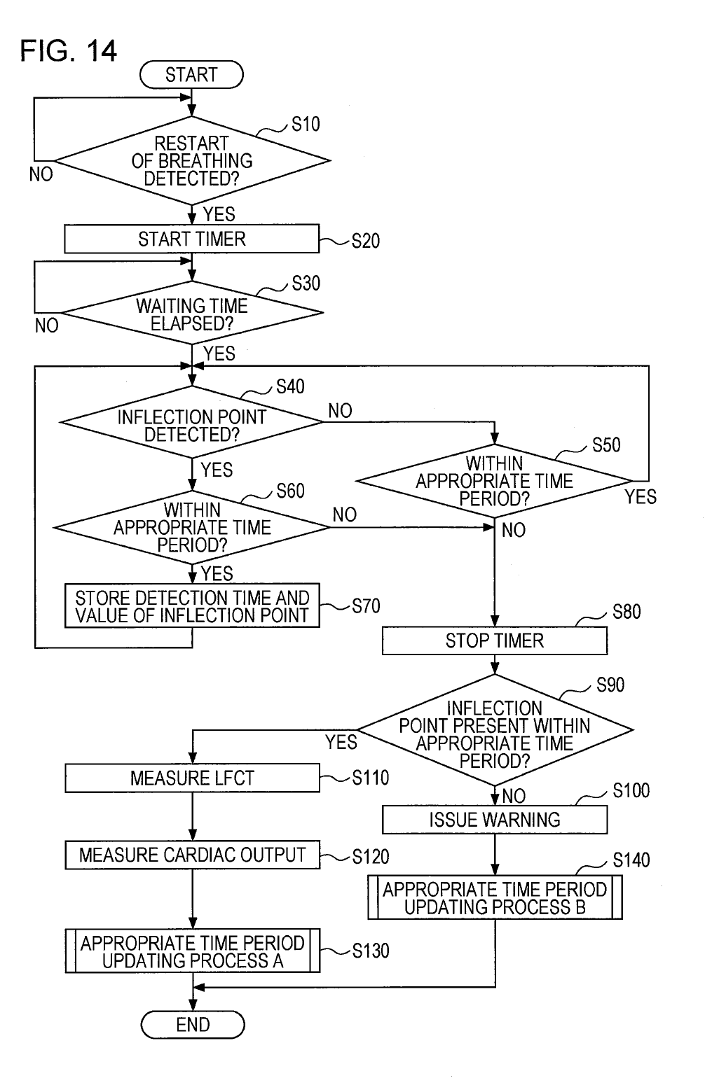

The process of the biological information measurement apparatus 10A is described in connection with FIG. 14.

FIG. 14 is a flowchart illustrating the biological information measurement process performed by the CPU 21 when a measurement instruction to measure the cardiac output is received from the user of the biological information measurement apparatus 10A via the input unit 27 with the optical sensor 11 mounted on the tip of a finger of the subject. Upon receiving the measurement instruction to measure the cardiac output, the biological information measurement apparatus 10A starts and continues to measure the oxygen saturation of the subject at least until the end of the measurement of the cardiac output.

The biological information measurement program describing the biological information measurement process is pre-stored on the ROM 22. The CPU 21 in the biological information measurement apparatus 10A reads the biological information measurement program from the ROM 22, and performs the biological information measurement process. The start time and end time of the appropriate time period are pre-stored on the non-volatile memory 24.

The flowchart of FIG. 14 is different from the flowchart of the biological information measurement process of the first exemplary embodiment illustrated in FIG. 11 in that the flowchart of FIG. 14 additionally includes steps S130 and S140. The rest of the flowchart of FIG. 14 is identical to the flowchart of FIG. 11.

If the inflection point of the oxygen saturation is not detected during the set appropriate time period, the CPU 21 performs an appropriate time period updating process B in step S140 subsequent to step S100 in which a warning is issued.

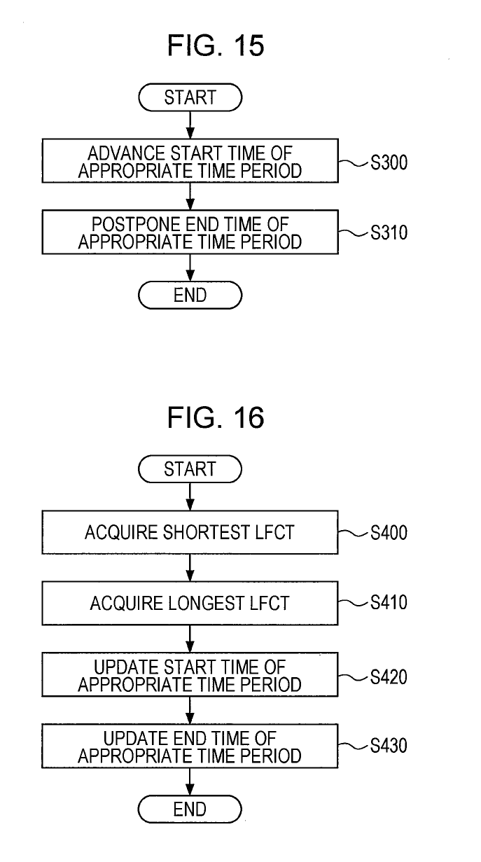

FIG. 15 is a flowchart illustrating the detail of the appropriate time period updating process B in step S140.

The fact that the appropriate time period updating process B is performed means that the inflection point appears outside the presently set appropriate time period. In step S300, the CPU 21 updates the appropriate time period such that the start time of the presently set appropriate time period is advanced. There is no particular limit to how long the start time of the presently set appropriate time period is to be advanced. A predetermined correction value M by which the start time of the present set appropriate time period is to be advanced may be a predetermined value.

In step S310, the CPU 21 updates the appropriate time period such that the end of the presently set appropriate time period is delayed. There is no particular limit to how long the end time of the presently set appropriate time period is to be delayed. The predetermined correction value M by which the end time of the presently set appropriate time period is to be delayed may be a predetermined value.

The appropriate time period updating process B in step S140 of FIG. 14 is thus complete.

Through the appropriate time period updating process B, the length of the appropriate time period is set to be longer than the length of the unmodified appropriate time period. The inflection point appearing in response to the restart of breathing of the subject and unable to be detected during the unmodified appropriate time period is more likely to be detected. More specifically, the modified appropriate time period has a more appropriate length than the unmodified appropriate time period such. As a result, LFCT is measured at a time and that measurement process of LFCT is applicable to more subjects.

When the inflection point of the oxygen saturation is detected within the presently set appropriate time period, the measurement of LFCT is performed in step S110 of FIG. 14. After the cardiac output is measured in step S120, an appropriate time period updating process A is performed in step S130.

FIG. 16 is a flowchart illustrating the detail of the appropriate time period updating process A.

In step S400, the CPU 21 reads all past LFCTs detected and stored on the non-volatile memory 24, namely, all the detection times t₂ of the past standard inflection points. From among the read detection times t₂, the CPU 21 acquires the earliest detection time t₂. That earliest time t₂ indicates the minimum value of LFCT of the subject up until now.

In step S410, the CPU 21 the CPU 21 acquires the latest detection time t₂ from among the read detection times t₂. That latest detection time t₂ indicates the maximum value of LFCT of the subject up until now.

In step S420, the CPU 21 updates the start time of the appropriate time period such that the detection time t₂ acquired in step S400 is the start time of a new appropriate time period.

In step S430, the CPU 21 updates the end time of the appropriate time period such that the detection time t₂ acquired in step S410 is the end time of the new appropriate time period.

The appropriate time period updating process A in step S130 of FIG. 14 is thus complete.

Through the appropriate time period updating process A, the length of the appropriate time period extended in the appropriate time period updating process B is shortened to a range that includes the standard inflection points detected in the past.

More specifically, by repeating the measurement of the cardiac output, the biological information measurement apparatus 10A autonomously sets the appropriate time period such that the appropriate time period includes any inflection point corresponding to the restart of breathing of the subject.

The disclosure is applicable to the real-time process in which the inflection point is successively detected while the oxygen saturation is being measured. The disclosure is also applicable to another process. For example, the oxygen saturation measured in response to the measurement instruction to measure the cardiac output is stored first on the non-volatile memory 24. After the measurement of the oxygen saturation is complete, the values of the oxygen saturation are read from the non-volatile memory 24. The inflection point of the oxygen saturation during the appropriate time period is detected.

In each of the exemplary embodiments, the biological information measurement process is implemented in software. The processes illustrated in FIGS. 11, 12, and 14 through 16 may be implemented in hardware on an application specific integrated circuit (ASIC). In such a case, a fast measurement process may be implemented.

In each of the exemplary embodiments, the biological information measurement program is installed on the ROM 22. The disclosure is not limited to this configuration. The biological information measurement program of the disclosure may be supplied in the recorded form on a computer readable recording medium. For example, the biological information measurement program of the disclosure may be supplied in the recorded form on one of optical discs including a compact disc ROM (CD-ROM), and a digital versatile disc ROM (DVD-ROM). The biological information measurement program of the disclosure may be supplied in the recorded form on one of semiconductor memories, including a universal serial bus (USB) memory and a flash memory. The biological information measurement apparatus 10 or 10A may obtain the biological information measurement program of the disclosure from an external apparatus connected to the communication system via the communication unit 29.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A biological information measurement apparatus comprising:
a first measurement unit that measures a value representing oxygen concentration in blood of a subject; and
a second measurement unit that measures, by referring to a change in the value measured by the first measurement unit, as an oxygen circulation time within a predetermined time period with an end time thereof set to be later than a restart time of breathing of the subject from holding of breathing, a time duration from the restart time to a detection time of an inflection point of the value detected after the restart time of breathing.

2. The biological information measurement apparatus according to claim 1, further comprising:
a reception unit that receives an instruction from a user; and
a modification unit that modifies at least one of a start time and the end time of the predetermined time period in response to the instruction received by the reception unit.

3. The biological information measurement apparatus according to claim 2, wherein the modification unit is configured to modify the start time of the predetermined time period to be later than the restart time.

4. The biological information measurement apparatus according to claim 2, wherein the modification unit is configured to set at least one of the start time and the end time of the predetermined time period on each subject.

5. The biological information measurement apparatus according to claim 3, wherein the modification unit is configured to set at least one of the start time and the end time of the predetermined time period on each subject.

6. The biological information measurement apparatus according to claim 2, further comprising a notification unit that issues a warning if a length of the predetermined time period modified by the modification unit is not appropriate for measuring the oxygen circulation time.

7. The biological information measurement apparatus according to claim 3, further comprising a notification unit that issues a warning if a length of the predetermined time period modified by the modification unit is not appropriate for measuring the oxygen circulation time.

8. The biological information measurement apparatus according to claim 4, further comprising a notification unit that issues a warning if a length of the predetermined time period modified by the modification unit is not appropriate for measuring the oxygen circulation time.

9. The biological information measurement apparatus according to claim 5, further comprising a notification unit that issues a warning if a length of the predetermined time period modified by the modification unit is not appropriate for measuring the oxygen circulation time.

10. The biological information measurement apparatus according to claim 1, further comprising an updating unit that updates a length of the predetermined time period in accordance with each of the oxygen circulation times measured by the second measurement unit such that the detection time of the inflection point of the value falls within the predetermined time period.

11. The biological information measurement apparatus according to claim 10, wherein the updating unit updates at least one of a start time and the end time of the predetermined time period on each subject in accordance with the oxygen circulation time of the subject measured by the second measurement unit.

12. The biological information measurement apparatus according to claim 1, wherein the second measurement unit measures the oxygen circulation time by detecting the inflection point of the value throughout the predetermined time period.

13. The biological information measurement apparatus according to claim 2, wherein the second measurement unit measures the oxygen circulation time by detecting the inflection point of the value throughout the predetermined time period.

14. The biological information measurement apparatus according to claim 3, wherein the second measurement unit measures the oxygen circulation time by detecting the inflection point of the value throughout the predetermined time period.

15. The biological information measurement apparatus according to claim 1, wherein the second measurement unit measures the oxygen circulation time using the detection time of the inflection point of the value falling within the predetermined time period out of the inflection points of the value detected during a time duration throughout which the first measurement unit measures the value.

16. The biological information measurement apparatus according to claim 1, wherein if a plurality of inflection points of the value has been detected during the predetermined time period, the second measurement unit measures as the oxygen circulation time a time duration from the detection time of the inflection point to the restart time, the inflection point being detected after the restart time and being smaller in value than remaining inflection points from among the plurality of infection points.

17. A biological information measurement apparatus comprising:
a first measurement unit that measures a value representing oxygen concentration in blood of a subject;
a second measurement unit that measures, by referring to a change in the value measured by the first measurement unit, as an oxygen circulation time a time duration from a restart time of breathing of the subject from holding of breathing to a detection time of an inflection point of the value; and
a modification unit that modifies at least one of a start time and an end time of a detection time period within which the inflection point of the value is detected.

18. The biological information measurement apparatus according to claim 17, further comprising a reception unit that receives an instruction from a user, wherein the modification unit modifies at least one of the start time and the end time of the detection time period in response to the instruction received by the reception unit.

19. The biological information measurement apparatus according to claim 18, wherein the modification unit modifies at least one of the start time and the end time of the detection time period on each subject.

20. A non-transitory computer readable medium storing a program causing a computer to execute a process for measuring biological information, the process comprising:
measuring a value representing oxygen concentration in blood of a subject; and
by referring to a change in the value measured, measuring as an oxygen circulation time within a predetermined time period with an end time thereof set to be later than a restart time of breathing of the subject from holding of breathing, a time duration from the restart time to a detection time of an inflection point of the value detected after the restart time of breathing.
